(19) United States
(12) Patent Application Publication
(10) Pub. No.: US 2018/0193527 A1
(43) Pub. Date: Jul. 12, 2018

(54) Biomimetic Scaffolds Including Devitalized Cells

(71) Applicant: University of South Carolina, Columbia, SC (US)

(72) Inventor: Esmaiel Jabbari, Bethesda, MD (US)

(21) Appl. No.: 15/861,124

(22) Filed: Jan. 3, 2018

Related U.S. Application Data
(60) Provisional application No. 62/444,468, filed on Jan. 10, 2017.

(51) Int. Cl. A61L 27/36; A61L 27/56; A61L 27/04; A61L 27/12; A61L 27/38; A61F 2/28

(52) U.S. Cl. CPC A61L 27/3695; A61L 27/56; A61L 27/047; A61L 2430/02; A61L 27/3604; A61L 27/3895; A61F 2/28; A61L 27/12

(57) ABSTRACT

Biomimetic scaffolds are described that incorporate devitalized cells that can function as a depot for the release of cell and tissue supporting factors. Methods for forming the biomimetic scaffolds are also described. The biomimetic scaffolds can be bone tissue biomimetic scaffolds and the devitalized cells of the scaffolds can serve as sustained release depots for osteogenic and bone-forming factors. The biomimetic scaffolds can be particularly beneficial as grafting materials for reconstruction of large (e.g., greater than about 0.5 mm³) skeletal injuries.

### CROSS REFERENCE TO RELATED APPLICATION

This application claims filing benefit of U.S. Provisional Patent Application Ser. No. 62/444,468 having a filing date of Jan. 10, 2017, entitled “Devitalized Cell Microsheets for Growth Factor Release and Immune Regulation,” which is incorporated herein by reference for all purposes.

### FEDERAL RESEARCH STATEMENT

This invention was made with Government support under Award Numbers CBET1403545 and IIP150024, awarded by the National Science Foundation and under Award Number AR063745, awarded by National Institute of Arthritis and Musculoskeletal and Skin Diseases of the National Institutes of Health. The Government has certain rights in the invention.

### BACKGROUND

Approximately 1.5 million Americans every year suffer from bone loss due to traumatic skeletal injuries, infection, and resection of primary tumors that require extensive grafting. Globally more than 2 million people every year require bone grafting to reconstruct and bridge the gap in large bone defects. For instance, more than 50,000 soldiers have been wounded by blast injuries from roadside bombs and high velocity guns with massive skeletal defects that require bone grafting and mechanical stabilization to prevent amputation, and the total number of spinal fusion procedures in the U.S. was 666,000 in the year 2014. Clearly, reconstruction of complex skeletal injuries due to trauma, infection, degeneration, or tumor resection presents a significant clinical problem.

Autograft bone is currently the gold standard in reconstruction because it provides a continuous supply of osteogenic and vasculogenic growth factors and cells within an osteoconductive matrix for healing without eliciting an autoimmune response. The superior regenerative capacity of autograft bone is attributed to the autogenic nature of the cells and secretion of a cocktail of cytokines from the autograft cells leading to the recruitment of osteoprogenitor and vasculogenic cells from the surrounding tissue to the injury site and induction of an anti-inflammatory response. Unfortunately, the quantity of autograft bone that can be harvested from a patient is insufficient to fill large skeletal defects. Further, surgery is required on an already injured patient for bone harvesting with associated donor site morbidity and surgical risks.

Processed allogeneic cadaver bone is used for bone grafting in orthopedic surgery when autograft bone is insufficient. In the US, over 0.8 million bone grafting procedures are performed annually using allograft tissue. Unfortunately, chemical processing of allograft bone with detergents to prevent immune response significantly reduces the amount of growth factors in the allograft. As a result, allogeneic cells and growth factors (e.g., vascular endothelial growth factor (VEGF), bone morphogenetic protein-2 (BMP2), etc.) that contribute to regeneration and healing are removed from allograft bone. There is also a risk of transmission of unknown pathogens with the use of processed allograft because tissue banks screen for only a limited number of known pathogens. Radiation treatment improves sterility but reduces osteoinductivity and fracture energy of allograft tissue. Demineralized bone matrix (DBM) lacks mechanical strength for large bone defects. In addition, osteoinductivity and biological activity of DBM is highly variable and dependent on the tissue of origin and processing conditions.

Even with such major shortcomings, allograft tissue is used extensively in orthopedic surgery because there is no viable alternative. Regrettably, the long-term failure rate of allograft bone implants in reconstruction of large bone defects is greater than 25%, mainly due to disease transmission, infection and non-union. In addition, allograft bone is recommended primarily for reconstruction of small (less than 0.5 mm³) defects due to insufficient mechanical stability, lack of vascularity, and inadequate resorption of the graft. As such, patients with traumatic large skeletal injuries undergo multiple costly operations followed by extensive rehabilitation to maintain proper bone alignment and length.

What are needed in the art are methods for producing implantable materials for reconstruction and implantable materials produced thereby. In particular, what are needed are implantable materials for reconstruction of skeletal defects of any size, and in particular for large skeletal defects.

### SUMMARY

According to one embodiment, disclosed is a biomimetic scaffold suitable for use as an implantable material, for instance in reconstruction applications, or as a cellular scaffold, for instance in bioengineering applications. A biomimetic scaffold can include a porous synthetic substrate and a plurality of devitalized cells incorporated in/on the substrate. For instance, the substrate can be a fibrous sheet-like substrate formed of a plurality of fibers, e.g., electrospun fibers, and the devitalized cells can include stem cells and/or differentiated cells that can function as a depot for sustained release of beneficial active agents, e.g., cytokines. The active agents can encourage growth and development of cells or tissue on/near the scaffold and, in the case of implantable scaffolds, can stimulate an anti-inflammatory constructive immune response in surrounding tissue. The biomimetic scaffold can be a bone tissue biomimetic material and can incorporate high levels of mineralization in conjunction with devitalized cells, e.g., human mesenchymal stem cells, endothelial colony forming cells, etc., so as to promote osteogenesis and/or osteoconductivity on/in the bone tissue biomimetic scaffold.

Also disclosed are methods for forming a biomimetic scaffold. For example, formation methods can include seeding living cells on a porous, synthetic substrate (e.g., a fibrous substrate in the form of a sheet), and culturing the cells on the substrate for a time to encourage adhesion and optionally differentiation of the cells. The method can also include devitalization of the cells to terminate activity of the cells without destroying the cellular components. For instance, the cells can be devitalized through lyophilization. Methods can optionally include addition of peptides to the porous substrate, e.g., to encourage mineralization of the substrate in formation of a bone tissue biomimetic.

### BRIEF DESCRIPTION OF THE DRAWINGS

FIG. 1A presents a scanning electron microscope (SEM) image of human mesenchymal stem cells (hMSCs) seeded on mineralized microsheets and cultured in basal DMEM medium for 1 day before devitalization.

FIG. 1B presents an SEM of hMSCs seeded on mineralized microsheets and cultured in basal DMEM medium for 1 day after devitalization.

FIG. 1C presents an SEM image of endothelial colony-forming cells (ECFCs) seeded on mineralized microsheets and cultured in vasculogenic medium for 7 days before devitalization.

FIG. 1D presents an SEM image of endothelial colony-forming cells (ECFCs) seeded on mineralized microsheets and cultured in vasculogenic medium for 7 days after devitalization.

FIG. 2A presents the cumulative total protein released from devitalized cell-seeded microsheets with incubation time in PBS.

FIG. 2B presents the cumulative total bone morphogenetic protein 2 (BMP2) released from devitalized cell-seeded microsheets with incubation time in PBS.

FIG. 2C presents the cumulative total vascular endothelial growth factor (VEGF) released from devitalized cell-seeded microsheets with incubation time in PBS.

FIG. 3A is an immuno-stained image of VEGF for ECFCs seeded on mineralized microsheets and differentiated in vasculogenic medium for 7 days.

FIG. 3B is an immuno-stained image of VEGF for hMSCs seeded on mineralized microsheets and cultured in basal DMEM medium for 1 day.

FIG. 3C is an immuno-stained image of BMP2 for ECFCs seeded on mineralized microsheets and differentiated in vasculogenic medium for 7 days.

FIG. 3D is an immune-stained image of BMP2 for hMSCs seeded on mineralized microsheets and cultured in basal DMEM medium for 1 day.

FIG. 4A provides the DNA content for hMSCs reseeded on devitalized cell-seeded mineralized microsheets and cultured in basal osteogenic medium for 21 days.

FIG. 4B provides the ALP activity for hMSCs reseeded on devitalized cell-seeded mineralized microsheets and cultured in basal osteogenic medium for 21 days.

FIG. 4C provides the calcium content for hMSCs reseeded on devitalized cell-seeded mineralized microsheets and cultured in basal osteogenic medium for 21 days.

FIG. 4D provides the mRNA expression of osteogenic marker ALP for hMSCs reseeded on devitalized cell-seeded mineralized microsheets and cultured in basal osteogenic medium for 21 days.

FIG. 4E provides the mRNA expression of osteogenic marker osteopontin for hMSCs reseeded on devitalized cell-seeded mineralized microsheets and cultured in basal osteogenic medium for 21 days.

FIG. 4F provides the mRNA expression of osteogenic marker osteocalcin for hMSCs reseeded on devitalized cell-seeded mineralized microsheets and cultured in basal osteogenic medium for 21 days.

FIG. 5A presents the alizarin red stained (dark) image of hMSCs seeded on mineralized microsheets and cultured in basal DMEM medium for 21 days (negative control).

FIG. 5B presents the alizarin red stained (dark) image of hMSCs reseeded on devitalized hMSC-seeded mineralized microsheets and cultured in basal osteogenic medium for 21 days.

FIG. 5C presents the alizarin red stained (dark) image of hMSCs seeded on mineralized microsheets and cultured in basal osteogenic medium supplemented with BMP2 for 21 days.

FIG. 6A presents the mRNA expression of vasculogenic marker VE cadherin for ECFCs, with or without hMSCs, reseeded on devitalized cell-seeded mineralized microsheets and cultured in basal vasculogenic medium for 7 days.

FIG. 6B presents the mRNA expression of vasculogenic marker vWF for ECFCs, with or without hMSCs, reseeded on devitalized cell-seeded mineralized microsheets and cultured in basal vasculogenic medium for 7 days.

FIG. 6C presents the mRNA expression of vasculogenic marker CD31 for ECFCs, with or without hMSCs, reseeded on devitalized cell-seeded mineralized microsheets and cultured in basal vasculogenic medium for 7 days.

FIG. 6D presents CD31 protein expression for ECFCs, with or without hMSCs, reseeded on devitalized cell-seeded mineralized microsheets and cultured in basal vasculogenic medium for 7 days.

FIG. 6E presents bands that are representative western blots corresponding to CD31 protein expressions (plus reference protein β-actin).

FIG. 7A presents mRNA expression of macrophage marker TNFα for M1 phenotype for human macrophages reseeded on devitalized cell-seeded mineralized microsheets and cultured in basal macrophage medium for 7 days.

FIG. 7B presents mRNA expression of macrophage marker IL1β for M1 phenotype for human macrophages reseeded on devitalized cell-seeded mineralized microsheets and cultured in basal macrophage medium for 7 days.

FIG. 7C presents mRNA expression of macrophage marker CCR7 for M1 phenotype for human macrophages reseeded on devitalized cell-seeded mineralized microsheets and cultured in basal macrophage medium for 7 days.

FIG. 7D presents mRNA expression of macrophage marker CD206 for M2 phenotype for human macrophages reseeded on devitalized cell-seeded mineralized microsheets and cultured in basal macrophage medium for 7 days.

FIG. 7E presents mRNA expression of macrophage marker CCL18 for M2 phenotype for human macrophages reseeded on devitalized cell-seeded mineralized microsheets and cultured in basal macrophage medium for 7 days.

FIG. 7F presents mRNA expression of macrophage marker CCL22 for M2 phenotype for human macrophages reseeded on devitalized cell-seeded mineralized microsheets and cultured in basal macrophage medium for 7 days.

FIG. 8A presents a representative dot blot for the expression of M1 marker TNFα versus the expression of M2 marker CD206 for macrophages seeded on devitalized cell-seeded microsheets and cultured in basal macrophage medium. The experimental groups included the mineralized microsheets seeded with hMSCs, cultured in basal DMEM medium for 1 day, devitalized, and reseeded with macrophages and cultured in basal macrophage medium for 1 day.

FIG. 8B presents a representative dot blot for the expression of M1 marker TNFα versus the expression of M2 marker CD206 for macrophages seeded on devitalized cell-seeded microsheets and cultured in basal macrophage medium. The experimental groups included the mineralized microsheets seeded with hMSCs, cultured in basal DMEM medium for 1 day, devitalized, and reseeded with macrophages and cultured in basal macrophage medium for 7 days.

FIG. 8C presents a representative dot blot for the expression of M1 marker TNFα versus the expression of M2 marker CD206 for macrophages seeded on devitalized cell-seeded microsheets and cultured in basal macrophage medium. The experimental groups included the mineralized microsheets seeded with ECFCs, cultured in vasculogenic medium for 7 days, devitalized, reseeded with macrophages and cultured in basal macrophage medium for 1 day.

FIG. 8D presents a representative dot blot for the expression of M1 marker TNFα versus the expression of M2 marker CD206 for macrophages seeded on devitalized cell-seeded microsheets and cultured in basal macrophage medium. The experimental groups included the mineralized microsheets seeded with ECFCs, cultured in vasculogenic medium for 7 days, devitalized, reseeded with macrophages and cultured in basal macrophage medium for 7 days.

### DETAILED DESCRIPTION

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment.

Biomimetic scaffolds are described that incorporate devitalized cells that can function as a depot for the release of cell and tissue supporting factors. Methods for forming the biomimetic scaffolds are also described. In one embodiment, the biomimetic scaffolds can be bone tissue biomimetic scaffolds and the devitalized cells of the scaffolds can serve as sustained release depots for osteogenic and bone-forming factors. The biomimetic scaffolds can be utilized in in vitro or ex vivo applications in tissue engineering applications as well as in in vivo applications, for instance as bone grafts for skeletal reconstruction surgery. The biomimetic scaffolds can be particularly beneficial as grafting materials for reconstruction of large (e.g., greater than about 0.5 mm³) skeletal injuries. Beneficially, the scaffolds can be loaded with autogenic (derived from patient) devitalized cells, for instance when utilized as implants in reconstruction surgeries. As such, the implants carrying the autogenic devitalized cells can elicit a constructive immune response and continually release factors for healing.

The devitalized cells of the biomimetic scaffolds can serve as depots for sustained release of a cocktail of cytokines and growth factors including BMP2 and VEGF, among others. Moreover, the biomimetic scaffolds can serve as depots for useful factors without negatively affecting macrophage polarization. For instance, the biomimetic scaffolds can stimulate higher M2 macrophage polarization compared to M1 in conjunction with release of proteins that encourage osteogenesis and/or vasculogenesis in the local area.

Macrophages include a heterogeneous population of cells found in most tissues of the body. These cells are capable of performing a broad spectrum of functions. Macrophage phenotypes are classified along a continuum between the extremes of pro-inflammatory M1 macrophages and anti-inflammatory M2 macrophages. The seemingly opposing functions of M1 and M2 macrophages must be tightly regulated for an effective and proper response to foreign molecules or damaged tissue. M1 phenotypes secrete factors that elicit a pro-inflammatory response to recruit immune cells and initiate an immune response whereas M2 phenotype secretes anti-inflammatory cytokines that deactivate the immune response and initiate a reparative or healing response.

Pro-inflammatory M1 macrophages remove damaged cells from the injury site in the initial stage of bone repair. While necessary for the initial stages of tissue repair, an excessive M1 activation inhibits the healing of damaged tissue through excessive matrix degradation and inhibition of tissue regeneration. In contrast, anti-inflammatory M2 macrophages secrete cytokines in the later stages of healing to promote tissue formation and remodeling. As discussed further in the Examples section below, the disclosed biomimetic scaffolds incorporating devitalized cells can stimulate polarization of macrophages to M2 phenotype as the ratio of M2/M1 phenotypes increase over time. Without wishing to be bound to any particular theory, and assuming that the immune-regulatory cytokines released by the devitalized cells on the biomimetic scaffolds are similar to those of live cells, it is believed that transforming growth factor-β (TGF-β) signaling pathway and its cytokine mediators PGE2, IL-4, IL-10, and IL-6 are implicated in macrophage polarization to M2 phenotype by the devitalized cells on the biomimetic scaffolds. Other signaling pathways that may be involved in M2 polarization of macrophages by devitalized cells of the biomimetic scaffolds are the SMAD/PI3K/Akt/mTOR pathway via the release of BMP2 from the devitalized cells, IL-4 signaling via the release of VEGF, and Wnt/Ca² pathway by the release of calcium from mineralized microsheets.

The type of devitalized cell incorporated on the biomimetic scaffold can vary depending upon, e.g., the application of the scaffold (ex vivo, in vitro, in vivo), and the living cells or tissue that will be held on/in/near the scaffold during use. For example, the devitalized cells can include stem cells and/or differentiated cells and can be selected to provide a depot of particular healing or growth factors to the local area of the scaffold during use. In one embodiment, human mesenchymal stem cells (hMSCs) and/or human endothelial progenitor cells (hEPCs) can be seeded, cultured, and devitalized on a biomimetic scaffold. In another embodiment, human chondrocytes can be seeded, cultured, and devitalized on a biomimetic scaffold for regeneration of articular cartilage tissue. In another embodiment, human cardiac tenocytes and/or ligament cells can be seeded, cultured, and devitalized on a biomimetic scaffold for regeneration of tendon or ligamentous tissues. In another embodiment, human cardiomyocytes optionally in combination with cardiac fibroblasts can be seeded, cultured, and devitalized on a biomimetic scaffold for regeneration of cardiac tissue. In another embodiment, human neural cells can be seeded, cultured, and devitalized on a biomimetic scaffold for regeneration of peripheral nerve tissue. These are just a few examples of the types of cells can be seeded, cultured, and devitalized on the biomimetic scaffold.

In ex vivo or in vivo applications, autogenic cells derived from the subject for whom the scaffold is specifically designed can be utilized as the source for the devitalized cells of the biomimetic scaffold. For example, autogenic MSCs and ECFCs derived from a patient's bone marrow and/or peripheral blood can be seeded and cultured on a scaffold and then devitalized prior to implantation. The devitalized cells can serve as a depot for sustained release of a mixture of cytokines to induce osteogenic and vasculogenic differentiation of migrating cells and to stimulate an anti-inflammatory constructive immune response. As such, the biomimetic scaffold can mimic the regenerative capacity of autograft bone.

Transplanted cells do not appear to contribute to repopulation of the injured tissue, but the devitalized cells can secrete growth factors that serve as mediators for recruitment of autologous cells to the injury site from the surrounding tissue. Osteogenesis and vasculogenesis are coupled processes and cytokines released from hMSCs and ECFCs can synergistically enhance osteogenic and vasculogenic differentiation of hMSCs and ECFCs. In addition, cytokines secreted by MSCs and their combination with other cells can affect the state of polarization of macrophages which in turn affects angiogenesis and maturation of blood vessels in an implant area.

In one embodiment, the devitalized cells of the scaffold can include undifferentiated stem cells. Use of undifferentiated stem cells can provide high levels of desirable factors in the scaffold in some embodiments. For instance, a biomimetic scaffold that includes devitalized undifferentiated hMSCs can exhibit a high cumulative BMP2 and VEGF release compared to a similar scaffold that includes devitalized differentiated hMSCs. This is consistent with studies showing that MSCs at early stage of osteogenic differentiation secrete higher amounts of BMP2 than late stage of differentiation and that MSCs serve as trophic mediators for endothelial cells by secreting VEGF and basic fibroblast growth factor (bFGF) through paracrine signaling. Further, use of devitalized undifferentiated MSCs or MSCs at early stage of differentiation can provide osteoconductivity in the area of an implant as compared to utilization of MSCs at a later stage. It has been reported that VEGF secretion and up-regulation of angiogenic genes of MSCs decrease as the cells differentiate to the osteogenic lineage. As such, in some embodiments, it may be beneficial to include devitalized MSCs that are undifferentiated or at an early stage of differentiation.

Multiple different types of devitalized cells can be incorporated in/on the biomimetic scaffold. For instance, VEGF and BMP2 release from a biomimetic scaffold that includes both devitalized hMSCs and devitalized ECFCs can be higher than a similar biomimetic that includes only devitalized hMSCs. This is consistent with previous observations that cross-talk between osteoprogenitor cells and endothelial progenitor cells can enhance osteogenesis and angiogenesis.

The substrate that carries the devitalized cells can be a porous, synthetic substrate. A synthetic material can be utilized so as to prevent issues that can arise from use of processed natural materials (e.g., DBM). In one embodiment, the porous, synthetic substrate can be a fibrous substrate, one example of which is described in U.S. Pat. No. 9,314,549, to Jabbari, which is incorporated herein by reference.

For example, the porous, synthetic substrate can include a fibrous sheet that includes nanofibers. A porous, synthetic substrate can include biocompatible polymer that can be relatively low in molecular weight and that, in one embodiment, can be biodegradable. For instance, the biocompatible polymer can have a number average molecular weight between about 1000 Da and about 10,000 Da. This is not a requirement, however, and higher molecular weight polymers may be used in some embodiments.

By way of example, a porous synthetic substrate can be formed of a lactide-based polymer. For instance a polylactic acid formed via ring-opening polymerization of lactide monomer derived from lactic acid can be utilized. In other embodiments, commercially available polymers can be used. For example, poly(lactides) available from Polysciences, Inc., Natureworks, LLC, Cargill, Inc., Mitsui (Japan), Shimadzu (Japan), or Chronopol can be utilized.

A porous, fibrous, synthetic substrate can be formed according to an electrospinning process. In this embodiment, a solution including the biocompatible polymer can be electrospun to form a fibrous sheet including nanofibers that incorporate the biocompatible polymer. The solution can generally include a total polymer content of about 30% or less.

Optionally, the synthetic substrate can include more than one polymer. The additional polymers can depend upon the final application of the biomimetic scaffold. For instance, in forming an implantable scaffold, additional polymers can be biocompatible resorbable polymers. By way of example, a low molecular weight polylactic acid can be combined with a higher molecular weight polylactic acid homopolymer or copolymer and the solution can then be electrospun. A high molecular weight polymer can facilitate fiber formation during electrospinning.

The polymer(s) component of an electrospinning solution can generally have a glass transition temperature (Tg) of between about 50° C. and about 150° C., which is above physiological temperature and beneath thermal degradation temperature. This can be beneficial in those embodiments in which an electrospun sheet is to be further processed, e.g., by annealing.

As is known, an electrospinning process includes the application of an electrical field to the solution of the polymer, inducing a charge on the individual polymer molecules. The polymer solution can be held in a capillary tube by its surface tension at the air-surface interface. Upon application of an electric field, a charge and/or dipolar orientation will be induced at the air-surface interface that causes a force that opposes the surface tension. At critical field strength, the repulsive electrostatic forces will overcome forces due to the surface tension, and a jet of polymeric material will be ejected from the capillary tube. The jet is elongated and accelerated by the external electric field as it leaves the capillary tube. The trajectory of the jet can be controlled by applying an appropriately oscillated electrostatic field, allowing for directional control of the jet. As the jet travels in air, some of the solvent can evaporate, leaving behind charged polymer fibers that can be collected on a take-up reel. As the fibers are collected, the individual fibers may fuse, forming a fibrous sheet on the take-up reel. In addition, the polymer jet, after deposition on the collector, can also be further stretched by the tangential force produced by the rotation of the wheel and form aligned fibers on the edge of the wheel.

The critical field strength required to overcome the forces due to solution surface tension and form the jet will depend on many variables of the system. These variables include not only the particular polymers and solvents included in the solution, but also the polymer concentration and solution viscosity, as well as temperature of the system. In general, characterization of the jet formed, and hence characterization of the fibers formed, depends primarily upon solution viscosity, net charge density carried by the electrospinning jet and surface tension of the solution. The ability to form the small diameter fibers depends upon the combination of all of the various parameters involved. For example, electrospinning of lower viscosity solutions will tend to form beaded fibers, rather than smooth fibers.

The minimum polymer concentration of a solution to produce bead-free fibers is generally about 10 wt. %. Below the critical concentration, surface tension breaks the accelerating jet of fibers into droplets. Beneficially, the morphological structure of the electrospun nanofibers can have diameters similar to collagen fibers (e.g., about 50 to about 500 nanometers, with an average diameter of about 200 nanometers in one embodiment). An individual electrospun fibrous sheet can be from about one micrometer to about 40 micrometer thick.

Following formation, an electrospun fibrous sheet can be removed from the take-up reel and used as a biomimetic scaffold or further processed with other fibrous sheets or other components to form a biomimetic scaffold.

The synthetic, porous scaffold can include components that can improve one or more functional aspects of the scaffold. For instance, and as described in U.S. Pat. No. 9,314,549, previously incorporated herein by reference, a scaffold can incorporate peptides that can improve mineralization of the scaffold. Other peptides that can be incorporated in a scaffold can encourage adhesion of cells to the scaffold or provide any other useful function, examples of which are provided herein.

Natural biomineralization is mediated by extracellular matrix (ECM) proteins with amino acid sequences rich in acidic amino acid residues like glutamic acid or aspartic acid. Bone ECM proteins that are rich in acidic residues nucleate CaP crystallization by surface-immobilization on collagen fibrils. For instance, nucleation, growth and stabilization of CaP nanocrystals on collagen fibers in the bone matrix is mediated by ECM non-collagenous proteins such as bone sialoprotein (BSP), osteonectin (ON), osteopontin (OP) and osteocalcin (OC). Glutamic acid (GLU) or aspartic acid sequences ranging from 2-10 residues in these proteins regulate nucleation and growth of CaP crystals on collagen fibers. Accordingly, in one embodiment, the biomimetic scaffold can incorporate a peptide that is rich in acidic residues. For instance, a biomimetic scaffold can include peptides that incorporate from 2 to about 10 glutamic acid and/or aspartic acid residues.

A peptide incorporated in the biomimetic scaffold can include other amino acid residues as well as or alternative to those that can encourage mineralization of the scaffold. For instance, the sulfhydryl groups of the cysteine residue can be convenient for conjugating a peptide to a polymer and a peptide can therefore include one or more cysteine residues. Other amino acids can be included in a peptide depending upon the utilization of the materials. For instance, a peptide can include a lysine group to facilitate labeling for imaging or quantitative analysis or additional functionalization. A peptide can also include inert amino acids like glycine or alanine to change solubility of a polymer-peptide conjugate in a solvent, increase the fraction of peptide on the scaffold surface, or to increase flexibility of the peptide chain attached to the scaffold surface. In another embodiment, a scaffold can incorporate a peptide that can encourage adhesion of a cell or other materials to the scaffold.

A peptide can be attached to a biomimetic scaffold either during formation of the scaffold or following formation of the scaffold substrate. For example, when considering electrospinning formation methods, a peptide can be conjugated to the polymer prior to formation of the electrospinning solution and the formed fibrous sheet can thus include the peptide at the surface of the nanofibers. For example, a lactide-based polymer that has been terminated with an unsaturated double-bond such as an acrylamide group can be conjugated via a sulfhydryl functional group to a peptide that is terminated with a cysteine residue.

When forming a bone biomimetic scaffold, the substrate (e.g., a fibrous sheet either as-formed or following any post-formation processing) can be further treated to mineralize the surface of the substrate. For example, a fibrous substrate can be incubated in a solution that includes the desired ionic mineral species, primarily calcium and phosphate, and the desired minerals can nucleate directly on the fibers. For example, a fibrous sheet that incorporates a peptide that includes glutamic acid and/or aspartic acid residues can be incubated in a simulated body fluid (SBF) or a modified simulated body fluid (mSBF) that includes a mixture of calcium salts, phosphate salts, sodium chloride, potassium chloride, buffers, one or more organic acids, etc., and calcium phosphate crystals can nucleate from acidic amino acid residue directly on the surface of the nanofibers.

A scaffold can include a single structure, e.g., a single fibrous sheet, or a plurality of components, which can be the same or different from one another. For instance, in one embodiment, a plurality of fibrous sheets can be laminated to one another and utilized in a thick, multi-layer form. For instance, a plurality of single sheets can be stacked and heat treated, which can shrink and densify the sheets as well as cause the individual sheets to adhere to one another.

In one embodiment, a substrate, e.g., a single- or multi-layer fibrous sheet, can be shaped into a three dimensional scaffold construct. For instance, one or more 3D constructs can be fabricated by wrapping a single or multi-layer fibrous sheet around a mold. The mold can have the desired shape of the scaffold. Cylindrical molds can be utilized in one embodiment, as this shape can mimic that of the osteons of the cortical bone. A mold can be wrapped once or multiple times by a fibrous sheet, depending upon the size of construct to be formed.

Following wrapping of the mold with a fibrous sheet, the sheet can be heat treated at a temperature that is above the glass transition temperature and below the melting temperature of the nanofibers. The heat treatment can shrink and densify the sheet as well as cause the fibrous sheet to maintain the shape of the mold and fuse multiple layers surrounding the mold to one another.

One or a plurality of molded structures can then be utilized as a biomimetic scaffold. For instance, a plurality of formed fibrous scaffolds can be combined (e.g., layered and/or molded with one another) to form a biomimetic scaffold capable of load bearing.

In another embodiment, the biomimetic scaffold can be sized for use as a filler in a reconstruction application. For instance, single or multi-layer mineralized sheets can be chopped or ground and used as a filler in a reconstruction application. The size of individual pieces of a scaffold can vary depending upon the application. For example, mineralized fibrous single or multi-layer sheets can be ground to very small pieces and used as a bone filler or chopped to somewhat larger pieces and used as bone chips in reconstruction applications.

The biomimetic scaffold can be seeded with the cell types of interest prior to devitalization of the cells. In general, the scaffold can be seeded with cells following all scaffold formation steps, but this is not a requirement, and in some embodiments, it may be preferable to seed the scaffold with the cells and devitalize the seeded cells prior to final shaping of the scaffold (e.g., cutting to a particular size and/or shape).

In general, the cells can be cultured on the scaffold for a period of time following initial seeding to encourage adhesion to the scaffold. The cell culture conditions can vary depending upon the particular cell types involved, as is known in the art. For instance, hMSCs, hEPCs, or a combination of hMSCs and hEPCs can be cultured in basal medium for cell adhesion to the scaffold for a period of time of about one day or more.

The time and conditions for culturing the cells can also be controlled so as to develop the seeded cells to a desired stage at which they can serve as a depot for the desired factors. For instance, in those embodiments in which the seeded cells are stem cells, it may be beneficial to culture the cells to encourage differentiation to a particular cell type and stage so as to develop the desired protein expression products in the cells. By way of example, hMSCs and/or hEPCs can be differentiated to the osteogenic or vasculogenic lineage as desired by incubation in the appropriate medium. Of course, it may be preferred to utilized devitalized stem cells on the biomimetic scaffolds, in which case the seeded cells can be cultured for a period of time (e.g., about 1 day) in the absence of osteo-/vasculo-inductive factors

Following seeding and culturing, the cells on the scaffolds can be devitalized. Any devitalization process can be carried out that is capable of terminating active processing of the cells while preserving the morphology and protein components of the cells. For instance, the cultured cells can be devitalized by lyophilization (e.g., cryodessication), thermal processing (hyperthermic devitalization), or the like.

The biomimetic scaffolds seeded with the devitalized cells can be utilized as formed, for instance as an implant in a skeletal reconstruction application, or further processed (e.g., sized or combined with other materials) prior to use. For instance, the biomimetic scaffold including the devitalized cells can be seeded with live cells for study or implantation.

The biomimetic scaffold can be utilized in treatment of complex skeletal injuries, for instance those brought about as a result of combat, trauma, and resection of primary tumors in soldiers, veterans, and the civilian population. Similarly, patients who suffer from low back pain, vertebral fracture and tumors, spinal infection, spinal deformities and degenerative disc disease and need to undergo a spinal fusion surgery can also benefit from disclosed biomimetic scaffolds.

Bone grafts based on the described techniques and materials can transform bone graft technology for regeneration of complex bone defects from current standards utilizing allograft tissues to provide safe, infection-free, mechanically stable, osteoinductive, and vasculoinductive grafts that can ultimately be displaced by the patient's own tissue.

The present disclosure may be better understood with reference to the Example set forth below.

### EXAMPLE

Materials: Poly(D,L-lactide) (PDLLA) with intrinsic viscosity of 0.65 dL/g and weight average molecular weight (M_{w}) of 90 kDa was received from LACTEL (Cupertino, Calif.). L-lactide monomer (LA; >99.5% purity) was received from Ortec (Easly, S.C.). Diethylene glycol (DEG), sodium chloride (NaCl), potassium chloride (KCI), calcium chloride monohydrate (CaCl₂.H₂O), magnesium chloride hexahydrate (MgCl₂.6H₂0), sodium bicarbonate (NaHCO₃), and monosodium phosphate (NaH₂PO₄) were purchased from ThermoFisher Scientific (Waltham, Mass.). Rink Amide NovaGel™ resin and Fmoc-protected amino acids were purchased from EMD Biosciences (San Diego, Calif.) 1,1,1,3,3,3-hexafluoro-2-propanol (HFIP) was purchased from VWR (West Chester, Pa.). Tin (II) 2-ethylhexanoate (TOC), acryloyl chloride (AC), triethylamine (TEA), ethylenediaminetetraacetic acid disodium salt (EDTA), deuterated chloroform (99.8% deuterated), dimethyl sulfoxide (DMSO), paraformaldehyde, lipopolysaccharide (LPS), 4,6-diamidino-2-phenylindole (DAPI), Alizarin red, penicillin, and streptomycin were purchased from Sigma-Aldrich (St. Louis, Mo.). Interferon-γ (INF-γ), interleukin-4 (IL4), and IL13 were purchased from Peprotech (Rocky Hill, N.J.). Dichloromethane (DCM, Acros Organics, Pittsburg, Pa.) was dried by distillation over calcium hydride. Diethyl ether and hexane were obtained from VWR (Bristol, Conn.) and used as received. Fetal bovine serum (FBS) was received from Atlas Biologicals (Fort Collins, Colo.). QuantiChrom calcium and alkaline phosphatase (ALP) assays were purchased from Bioassay Systems (Hayward, Calif.). DMEM cell culture medium, Dulbecco's phosphate-buffer saline (PBS), trypsin-EDTA, Quant-it Pico-Green dsDNA reagent kit were received from Invitrogen (Carlsbad, Calif.). Human vascular endothelial growth factor (VEGF), recombinant human bone morphogenetic-2 (BMP2), related ELISA kits, and bicinchoninic acid (BCA1 assay) kit for determination of total protein were purchased from Sigma-Aldrich (St. Louis, Mo.). EGM-2 medium, human fibroblast growth factor-B (hFGF-B), R3-insulin like growth factor (IGF), human epidermal growth factor (hEGF), ascorbic acid (AA), β-sodium glycerophosphate (βGP), dexamethasone (DEX), hydrocortisone, gentamycin, and amphotericin B were purchased from Lonza (Hopkinton, Mass.). All forward and reverse primers were received from Integrated DNA technologies (Coralville, Iowa). All solvents were reagent grade and used as received without further purification. Human mesenchymal stem cells (hMSCs), harvested from the donor's posterior iliac crest, were received from Lonza (Allendale, N.J.). Human endothelial colony-forming cells (ECFCs), harvested from the donor's peripheral blood was received from Boston Children Hospital (Boston, Mass.). Human CRL-9850 macrophages harvested from spleen and Iscove's Modified Dulbecco's (IMDM, 30-2005) medium were received from American Type Culture Collection (ATCC, Manassas, Va.). Radioimmunoprecipitation assay (RIPA) lysis buffer with EDTA-free protease inhibitor cocktail (cOmplete®, Mini) was purchased from Roche Life Science (Indianapolis, Ind.).

Synthesis of mineralized fiber microsheets: Low molecular weight poly(DL-lactide) (LMPLA) was synthesized by ring-opening polymerization of LA monomer according to known methodology. LMPLA was reacted with AC to form acrylate-terminated LMPLA. Number average molecular weight (M_{N}) and polydispersity index (PI) of Ac-LMPLA, were determined from the chemical shifts in ¹H-NMR spectrum and found to be 5.3 and 1.2 kDa, respectively. Amino acid sequence Glu-Glu-Gly-Gly-Cys (GLU peptide) was synthesized manually on Rink Amide NovaGel™ resin. GLU peptide was conjugated to Ac-LMPLA by Michael addition reaction to produce a GLU-LMPLA conjugate. The average number of peptides per GLU-LMPLA conjugate was 1.3, determined from the chemical shifts in ¹H-NMR spectrum of the conjugate. GLU-LMPLA (1.5 wt. % based on total solution weight) and PDLLA (8 wt. %) were mixed in HFIP solvent and the mixture was electrospun to form aligned nanofiber microsheets. The polymer solution was injected into the field (20 kV) at the rate of 0.8 mL/h through a 21-gauge needle and collected on a rotating wheel with a rotation speed of 1800 rpm and needle-collector distance of 7.5 cm. The microsheets were immersed in a modified simulated body fluid (SFB) containing CaCl₂.H₂O, NaCl, NaH₂PO₄, MgCl₂.6H₂O and KCI for 12 h for nucleation and growth of calcium phosphate (CaP) nanocrysyals with GLU peptides serving as nucleation sites. CaP content of the microsheets after immersion in the modified SFB was 160±20 wt. % based on dry weight of the polymer, calcium to phosphate (Ca/P) ratio and crystallinity were 1.88±0.1 and 21±2%, respectively, measured by QuantiChrom calcium assay and energy-dispersive x-ray analysis (EDS). The average size of the nucleated CaP crystals on the microsheets was 210±15 nm measured from scanning electron microscope (SEM) images.

Cell culture: hMSCs were cultured in a high glucose DMEM medium supplemented with 10% FBS, 100 units/mL penicillin and 100 μg/mL streptomycin (basal medium) at seeding density of 5000 cells/mL. hMSCs with <5 passages (according to supplier's instruction) were enzymatically lifted with trypsin-EDTA after reaching 70% confluency and used for cell seeding on the microsheets. ECFCs as cobblestone-like cell colonies were cultured in full EGM-2 medium supplemented with 20% FBS on 1% gelatin-coated flasks at a density of 6500 cells/mL. ECFCs with <4 passages were enzymatically lifted with trypsin-EDTA and used for cell seeding on the microsheets. Human macrophages were cultured and expanded in IMDM medium supplemented with 10% FBS without the addition of cytokines (basal macrophage medium).

Cell devitalization on mineralized microsheets: Microsheets were sterilized by incubation in a solution containing penicillin G (10,000 U/mL), streptomycin (10,000 μg/mL) and amphotericin B (250 μg/mL) overnight followed by washing 3× with PBS. For MSC seeding, the sterilized microsheets were seeded with 2×10⁵ cells in 100 μL basal DMEM medium (DMEM medium supplemented with 10% FBS, 100 units/mL penicillin G, and 100 μg/mL streptomycin) or 2×10⁵ ECFCs in basal EGM-2 medium (complete EBM-2 medium supplemented with 20% FBS) and incubated for 24 h. For osteogenic differentiation of hMSCs, the medium was replaced with osteogenic medium (basal DMEM medium supplemented with 50 μg/mL AA, 10 mM βGP, and 100 nM DEX) and the cell-seeded microsheets were cultured for 21 days. For ECFC seeding, the microsheets coated with 1 wt. % gelatin solution were seeded with 2×10⁵ ECFCs in basal EGM-2 medium and incubated for 24 h. For vasculogenic differentiation of ECFCs, the medium was replaced with vasculogenic medium (basal EGM-2 medium supplemented with 25 ng/mL VEGF) and the microsheets were cultured for 7 days. For mixed MSC+ECFC seeding, the gelatin-coated microsheets were seeded with 2×10⁵ MSC+ECFC (50:50 MSC:ECFC) in basal EGM-2 medium, cultured in basal EGM-2 medium for 24 h, followed by cultivation in vasculogenic medium for 7 days. The hMSC- or ECFC-seeded microsheets were devitalized before (one day after cell seeding) and after (7 days in vasculogenic or 21 days in osteogenic medium) cultivation. For devitalization, medium was removed, microsheets were washed with PBS and lyophilized at −60° C.

Protein release from devitalized microsheets: The cell-seeded lyophilized microsheets were incubated in PBS at 37° C. (1 cm³ of the microsheet was placed in 1 mL PBS and the release medium was changed every day). At each time point (1, 4, and 7 days), microsheet samples were removed and the amount of BMP2 or VEGF as well as the total protein released was measured. The total protein was measured with BCA1 total protein assay according to manufacturer's instructions. The amount of BMP2 and VEGF was measured with their respective ELISA kits following the manufacturer's instructions.

Osteogenic and vasculogenic differentiation hMSCs and ECFCs reseeded on devitalized cell microsheets: The devitalized cell-seeded microsheets with significant release of BMP2 were selected for reseeding with hMSCs and culturing in basal osteogenic medium (osteogenic medium without DEX and BMP2) for 21 days to evaluate the effect of devitalized cells on osteogenesis. Negative and positive control groups for osteogenic experiments were mineralized microsheets seeded with hMSCs and cultured in basal osteogenic medium without and with BMP2 (100 ng/mL) supplementation, respectively. At each time point (7, 14, 21 days), the samples were evaluated for cell content, ALP activity, extent of mineralization, and expression of osteogenic markers ALP, osteopontin (OP), and osteocalcin (OC). The devitalized cell-seeded microsheets with significant release of VEGF were selected for reseeding with ECFCs, with (mixture of 50:50) or without hMSCs, and culturing in basal vasculogenic medium (vasculogenic medium without VEGF) for 7 days to evaluate the effect of devitalized cells on vasculogenesis. Negative and positive control groups for vasculogenic experiments were mineralized gelatin-coated microsheets seeded with ECFCs and cultured in basal vasculogenic medium without and with VEGF (25 ng/mL) supplementation, respectively. At each time point (1, 4, 7 days), the samples were evaluated by cell content and expression of vasculogenic markers VE-cadherin, von Willebrand factor (vWF), and PECAM1 (CD-31).

Alizarin red and immunohistochemical staining: The samples were fixed with 4% paraformaldehyde. The fixed samples were stained with Alizarin red. For immunostaining, the fixed samples were permeabilized and blocked. Next, the samples were incubated with BMP2 and VEGF antibodies (1:100 dilution) in PBS containing 1% BSA at ambient condition for 1 h. After PBS washing, the samples were incubated in the blocking solution for 1 h followed by incubation in the secondary antibody solution (bovine anti-rabbit IgG-FITC, 1:100 dilution) in the dark for 1 h. Then, the samples were counterstained with phalloidin and DAPI to image the actin filaments and nuclei of the cells. Secondary antibodies without the primaries were used as negative control. Stained samples were imaged with a Nikon Eclipse Ti-ε inverted fluorescent microscope.

Biochemical analysis: At each time point (7, 14 and 21 days), the MSC-reseeded devitalized microsheets were incubated in serum-free DMEM for 8 h to remove serum proteins, washed with PBS, homogenized in lysis buffer, and sonicated to rupture the cell membrane. DNA content, ALP activity, and calcium content of the samples were quantified with PicoGreen DNA assay, QuantiChrom ALP assay, and QuantiChrom calcium assay, respectively, according to the manufacturer's instructions. ALP activity as well as calcium content of each sample was normalized by dividing to its corresponding DNA content.

mRNA analysis: Total RNA of each cell-seeded microsheet sample was extracted using TRIzol plus RNeasy Mini-Kit (Qiagen, Valencia, Calif.) and genomic DNA was removed with Deoxyribonuclease I (Invitrogen) according to the manufacturer's instructions. 250 ng total RNA quantified by an ND-1000 NanoDrop spectrophotometer (ThermoFisher) was subjected to cDNA synthesis using a Promega reverse transcription kit (Madison, Wis.) and amplification by rt-qPCR using SYBR green RealMasterMix (Eppendorf) in a Bio-Rad CXF96 PCR system (Hercules, Calif.) for the expression of osteogenic and vasculogenic genes. Primers for osteogenic genes ALP (early marker), osteopontin (OP, late marker), and osteocalcin (OC, terminal osteogenic differentiation marker), and those for vasculogenic markers VE-cadherin (early marker), vWF (late marker), and PECAM1 (late marker) were designed by Primer3 web-based software. To compare expressions between experimental groups, mRNA fold difference in expression of the gene of interest was calculated by normalizing the expression to that of GAPDH house-keeping gene followed by normalizing against day zero expression.

Western blot analysis: At each time point (4, 7 and 10 days), protein content of the cell seeded microsheets were extracted by homogenization in RIPA lysis buffer according to manufacturer's instructions and the expression of CD31 protein was quantified. Briefly, the extracted proteins were separated by running the extract through a 7.5% SDS-PAGE electrophoresis gel (10 μg protein per well) using the Mini-gel system (Bio-Rad). The pattern of separated proteins was transferred to a nitrocellulose membrane. After blocking (Blotto solution, Santa Cruz Biotechnology), the membranes were incubated with a mixture of primary rabbit anti-human antibodies (CD31 and β-actin; 1:10,000 dilution) in PBS with 5% dry milk and 0.1% Tween-20 overnight at 4° C. After washing, the membranes were incubated with horseradish peroxidase-conjugated secondary antibodies (1:5000) for 1 h, washed, and incubated with enhanced luminol-based (ECL) detection reagent (Santa Cruz Biotechnology). The luminescent intensity of antibody-antigen bands, captured using a ChemiDoc MP system (Bio-Rad) was quantified with Image-J software (National Institutes of Health, Bethesda, Md.).

Macrophage polarization and flow cytometry: For evaluation of immune response, the devitalized cell-seeded microsheets were reseeded with human macrophages (from a solution with 10⁶ cells/mL) and cultured in basal macrophage medium (IMDM medium supplemented with 10% FBS). At each time point (1, 4, 7 days), macrophages were gently collected from the surface of microsheets with a cell scraper and evaluated with respect to M1 versus M2 polarization by gene expression and flow cytometry. Gene expression analysis was performed with rt-qPCR using surface markers IL1β, CCR7 and TNFα for M1 and CD206, CCL18 and CCL22 for M2 polarization. Differential gene expressions were reported as the ratio of expression in macrophages seeded on devitalized cell-seeded microsheets to the expression of macrophages prior to seeding on the microsheets (seeded on culture plates and incubated in basal macrophage medium, M0 control). For fluorescence activated cell sorting (FACS), the collected macrophages were fixed in 4% paraformaldehyde for 1 hour, centrifuged, and the cell pellet was resuspended in FACS Permeabilizing Solution™ (BD Biosciences, Franklin Lakes, N.J.) for 10 min at ambient condition, centrifuged and washed with cold PBS containing 5% BSA. The fixed macrophages were incubated with phycoerythrin (PE) mouse anti-human TNFα and fluorescein isothiocyanate (FITC) mouse anti-human CD206 (BD Biosciences, Franklin Lakes, N.J.) in PBS with 5% BSA for 45 min on ice in dark. The immune-stained macrophages were washed with cold PBS containing 5% BSA and analyzed by a flow cytometer (FC500, Beckman Coulter, Brea, Calif.).

Electron microscopy: The devitalized cell-seeded microsheets as well as cell-reseeded microsheets were imaged with a Tescan VEGA3 SBU variable pressure scanning electron microscope (SEM, Kotoutovice, Czech Republic) with an accelerating voltage of 8 KeV. Samples were coated with gold using a Denton Desk II sputter coater (Moorestown, N.J.) at 20 mA for 75 sec.

Statistical analysis: All experiments were performed in triplicate and expressed as means±standard deviation (SD). Significant differences between groups were determined using a two-way ANOVA with replication test, followed by a two-tailed Students t-test. P values less than 0.05 were considered statistically significant.

### Results

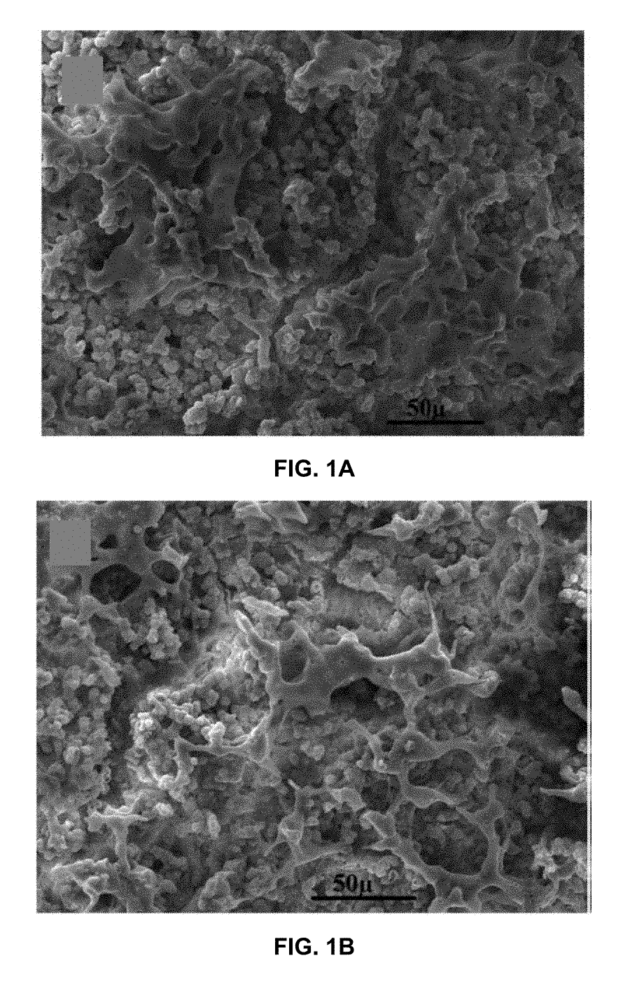

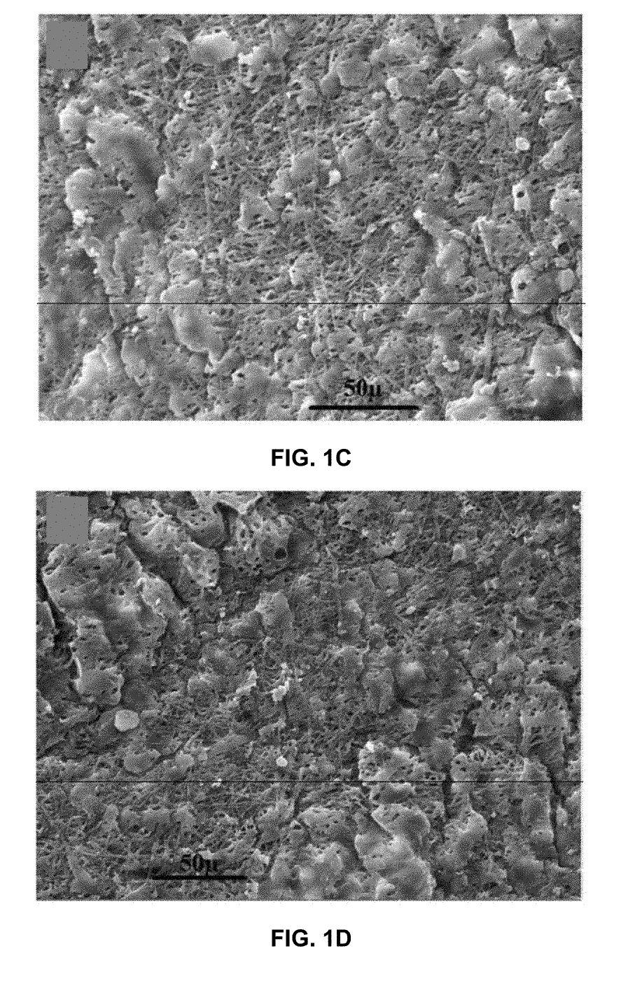

The SEM images in FIG. 1A-FIG. 1D show the effect of devitalization by freeze-drying on integrity and shape of hMSCs or ECFCs seeded on mineralized microsheets. FIG. 1A and FIG. 1B show the hMSCs cultured in basal DMEM for 1 day before (FIG. 1A) and after (FIG. 1B) devitalization and FIG. 1C and FIG. 1D show the ECFCs cultured in vasculogenic medium for 7 days before (FIG. 1C) and after (FIG. 1D) devitalization. As shown, the cells remained intact after devitalization irrespective of cell type or culture condition.

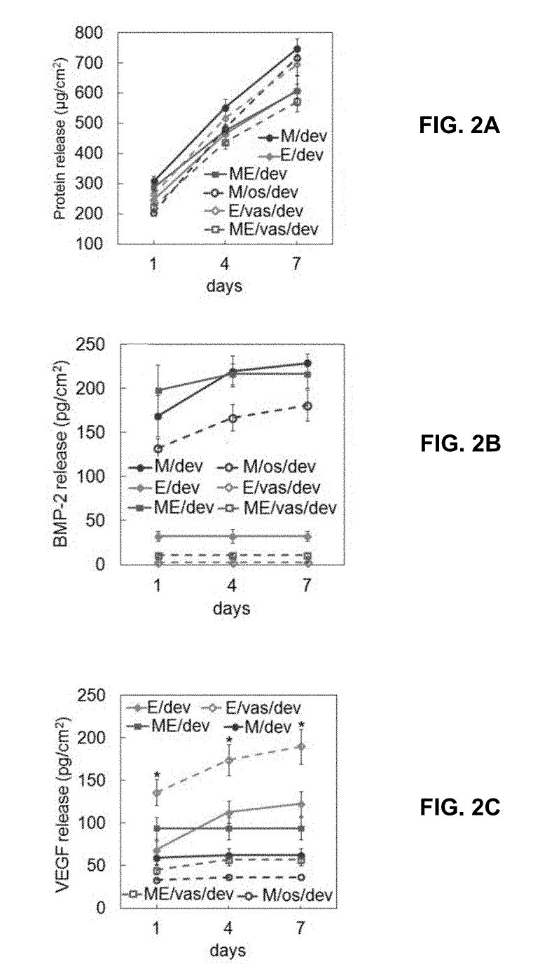

FIG. 2A, 2B, and 2C present the total protein (FIG. 2A), total BMP2 (FIG. 2B) and total VEGF (FIG. 2C) released from devitalized cell-seeded microsheets over the incubation time. The groups on the figures include: hMSCs seeded on mineralized microsheets and cultured in basal DMEM medium for 1 day and devitalized (M/dev); seeded hMSCs differentiated in osteogenic medium for 21 days and devitalized (M/os/dev); ECFCs seeded on the microsheets and cultured in basal EGM-2 medium for 1 day and devitalized (E/dev); seeded ECFCs differentiated in vasculogenic medium for 7 days and devitalized (E/vas/dev); hMSCs+ECFCs (1:1 mixture) seeded on the microsheets and cultured in basal EGM-2 medium for 1 day and devitalized (ME/dev); and seeded hMSCs+ECFCs cultured in vasculogenic medium for 7 days and devitalized (ME/vas/dev). The asterisk in (FIG. 2C) indicates a statistically significant difference (P<0.05) between the test group and all other groups for the given time point. Error bars correspond to means±1 SD for n=3.

As shown in FIG. 2A, all cell sheets regardless of cell type or culture condition showed similar rates of total protein release with incubation time. After 7 days of incubation, the seeded hMSCs cultured in basal DMEM medium for 1 day prior to devitalization (M/dev) had the highest amount of released proteins whereas the seeded hMSCs+ECFCs cultured in vasculogenic medium for 7 days prior to devitalization (ME/vas/dev) had the lowest released proteins. Cell differentiation prior to devitalization slightly decreased the amount of released proteins for hMSCs and hMSCs+ECFCs after 7 days, whereas ECFC differentiation slightly increased the amount released.

As shown in FIG. 2B, hMSC and hMSC+ECFC microsheets without differentiation had the highest release of BMP2 after devitalization. hMSCs differentiated to osteogenic lineage prior to devitalization (M/os/dev) showed significant release of BMP2 but the amount was less than that of undifferentiated hMSCs (M/dev). Among all groups, only the undifferentiated hMSC sheets showed steady release of BMP2 after devitalization with incubation time. Conversely, ECFC (E/vas/dev) and hMSC+ECFC (ME/vas/dev) microsheets differentiated to vasculogenic lineage prior to devitalization showed no BMP2 release. In general, cell differentiation led to a reduction in the amount of BMP2 released with incubation time from devitalized cell sheets.

Cumulative release of VEGF from the devitalized cell sheets with incubation time in PBS is shown in FIG. 2C. In contrast to BMP2 release, ECFC (E/vas/dev) seeded microsheets after differentiation to vasculogenic lineage showed highest and sustained release of VEGF with incubation time after devitalization. Undifferentiated ECFC cell sheets (E/dev) showed significant release of VEGF after devitalization but the amount was less than that of differentiated ECFCs (E/vas/dev). Conversely, hMSC (M/os/dev) and hMSC+ECFC (ME/vas/dev) microsheets after differentiation showed no VEGF release after devitalization.

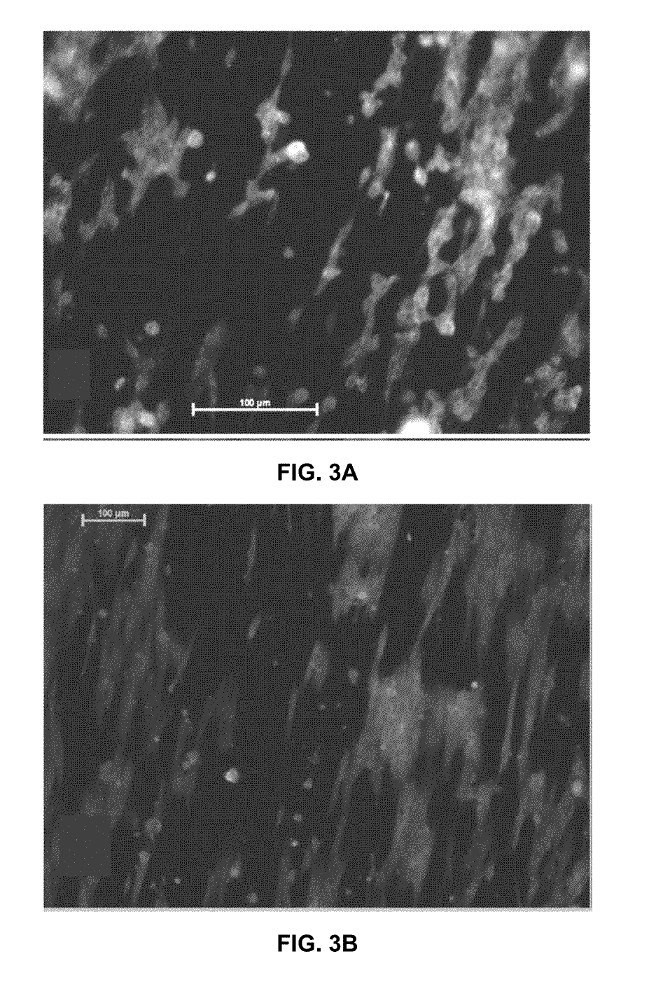

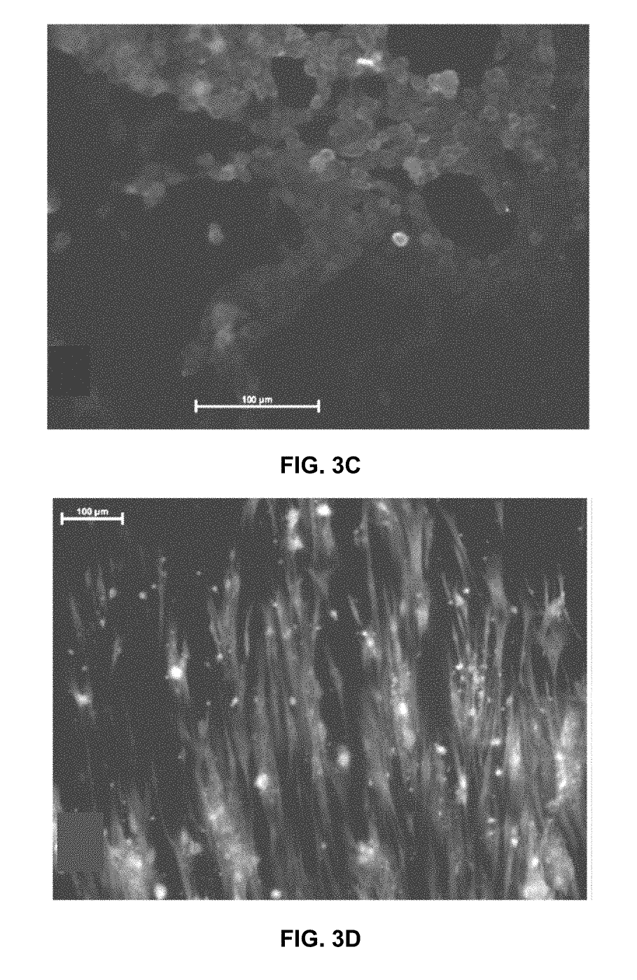

BMP2 and VEGF immuno-staining images of devitalized cell-seeded microsheets are shown in FIG. 3A-FIG. 3D. ECFCs seeded on the microsheets and differentiated to vasculogenic lineage stained for VEGF (FIG. 3A, lighter) but not BMP2 (FIG. 3C). Conversely, undifferentiated hMSCs seeded on the microsheets showed slight staining for VEGF (FIG. 3B) and strong staining for BMP2 (FIG. 3D). The results in FIG. 2A-2C and 3A-3D demonstrated that undifferentiated hMSC and vasculogenic-differentiated ECFC microsheets had the highest sustained release of BMP2 and VEGF, respectively, after devitalization.

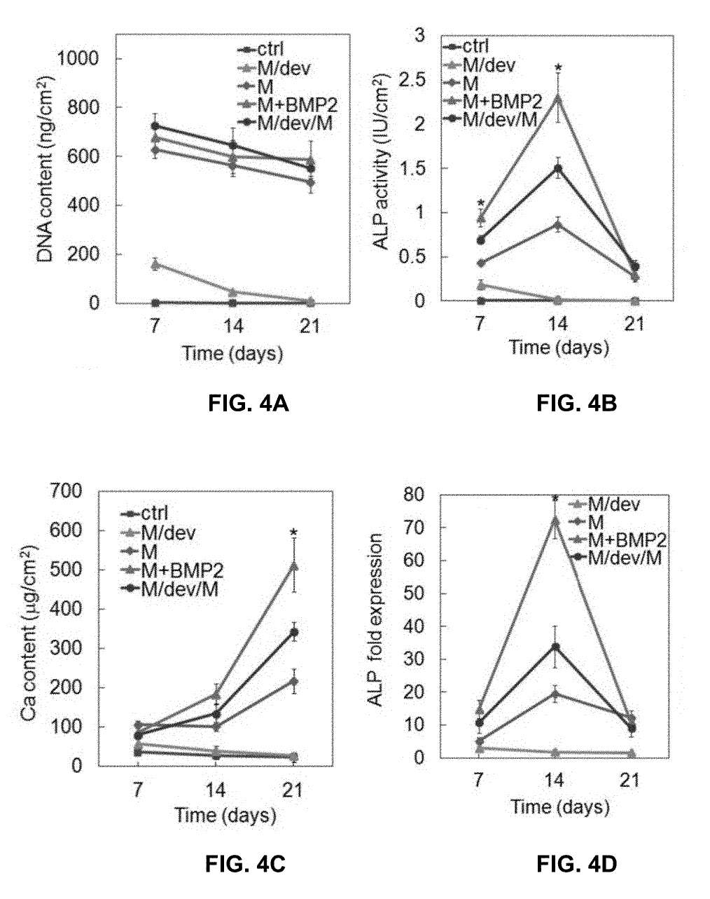

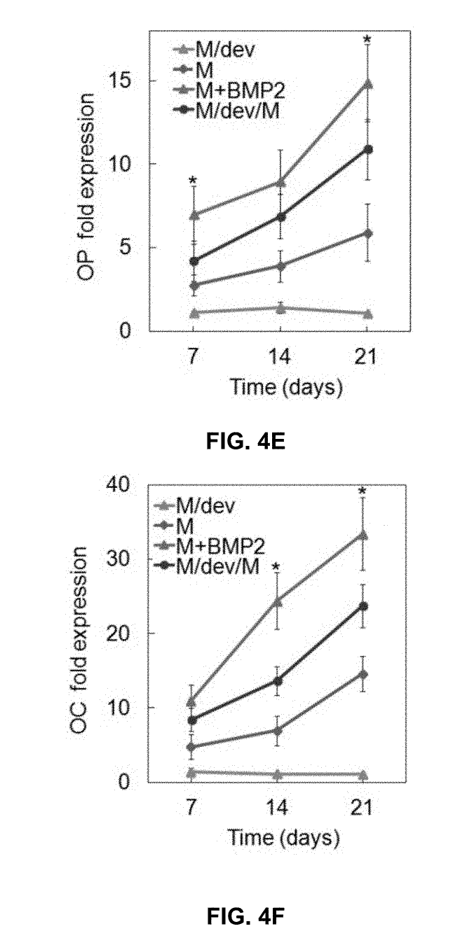

The effect of devitalized cells on osteogenic differentiation was evaluated by reseeding hMSCs on devitalized cell sheets and culturing in basal osteogenic medium (osteogenic medium without DEX and BMP2) for 21 days. Results are shown in FIG. 4A-FIG. 4F. Groups in the figures included mineralized microsheets without cells cultured in basal osteogenic medium (ctrl, negative control); microsheets seeded with hMSCs, cultured in basal DMEM medium for 1 day, devitalized followed by culturing (without cell reseeding) in basal osteogenic medium (M/dev, control); microsheets seeded with hMSCs and cultured in basal osteogenic medium without (M, control) and with (M+BMP2, positive control) BMP2 supplementation; and microsheets seeded with hMSCs, cultured in basal DMEM medium for 1 day, devitalized, reseeded with hMSCs and cultured in basal osteogenic medium (M/dev/M, experimental group). An asterisk in (FIG. 4B-FIG. 4F) indicates a statistically significant difference (P<0.05) between the test group and all other groups for the given time point. Error bars correspond to means±1 SD for n=3.

DNA content of the devitalized cell sheets without cell reseeding decreased with incubation time due to rupture and fragmentation of the devitalized cells (FIG. 4A). DNA content of the microsheets with live hMSCs (M, M/dev/M, M+BMP2) was much higher than the devitalized cell microsheets (M/dev) and DNA content decreased slightly with incubation time. The devitalized cell sheets without cell reseeding had very low ALP activity and calcium content with a decreasing trend with incubation time mainly due to the loss of devitalized cells.

ALP activity and calcium content of the microsheets with live hMSCs (M, M/dev/M, M+BMP2) significantly increased with incubation time (FIG. 4B, FIG. 4C). ALP activity of hMSC seeded microsheets cultured in basal osteogenic medium (M), hMSCs seeded on devitalized cell sheets and cultured in basal osteogenic medium (M/dev/M), and hMSC seeded microsheets cultured in basal osteogenic medium supplemented with BMP2 (M+BMP2) peaked at 0.9, 1.5, and 2.3 IU/cm², respectively, after 14 days of incubation; calcium content of the aforementioned groups were 220, 340, and 510 μg/cm² after 21 days. Although the ALP activity and calcium content of the devitalized cell microsheets reseeded with hMSCs and cultured in basal osteogenic medium (experimental group, M/dev/M) was lower than the hMSC seeded micrsosheets cultured in basal osteogenic medium supplemented with BMP2 (positive control, M+BMP2), ALP and calcium content of the experimental group was significantly higher than the hMSC seeded microsheets cultured in basal medium (negative control, M). The peak ALP activity and calcium content of hMSCs cultured on devitalized cell sheets in basal osteogenic medium were 1.7 and 1.6 fold higher than those hMSCs cultured on mineralized sheets without devitalized cells, respectively, but 34% and 33% lower than those hMSCs cultured on mineralized sheets in basal osteogenic medium supplemented with BMP2.

mRNA expressions for ALP, OP and OC (FIG. 4D-4F) of the hMSC seeded microsheets, with or without devitalization, were consistent with ALP biochemical activity and calcium content. For example, the peak OP fold mRNA expression for M/dev (negative control), M (negative control), M/dev/M (experimental group), and M+BMP2 (positive control) was 1.5, 6, 11, and 15, respectively.

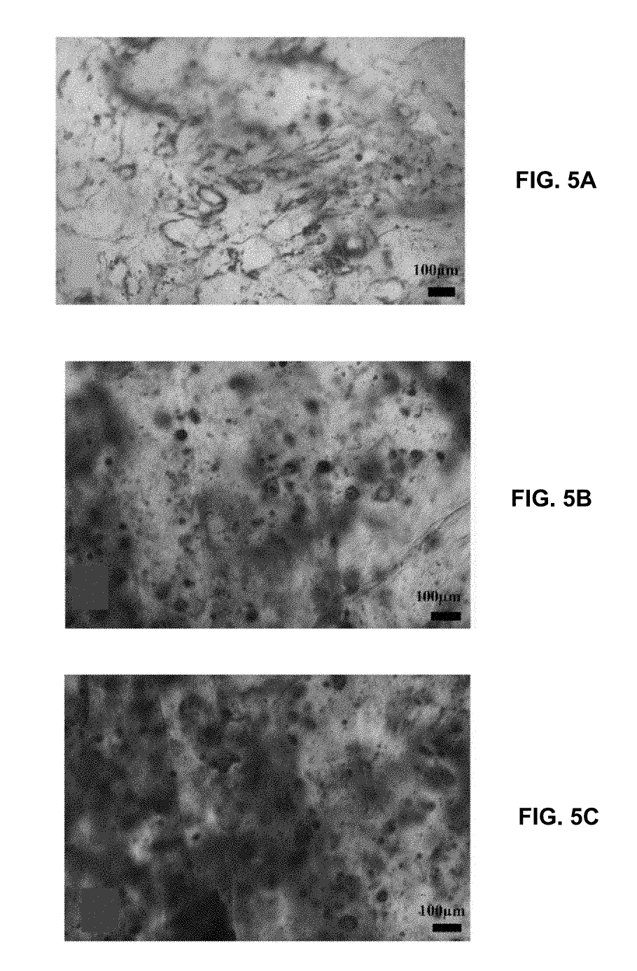

The intensity of Alizarin red staining for mineralized ECM production by hMSCs seeded on devitalized cell sheets and cultured in basal osteogenic medium (FIG. 5B) was higher than hMSCs seeded on microsheets without devitalized cells and cultured in basal osteogenic medium (FIG. 5A) but lower than hMSC seeded microsheets without devitalized cell and cultured in basal osteogenic medium supplemented with BMP2 (FIG. 5C).

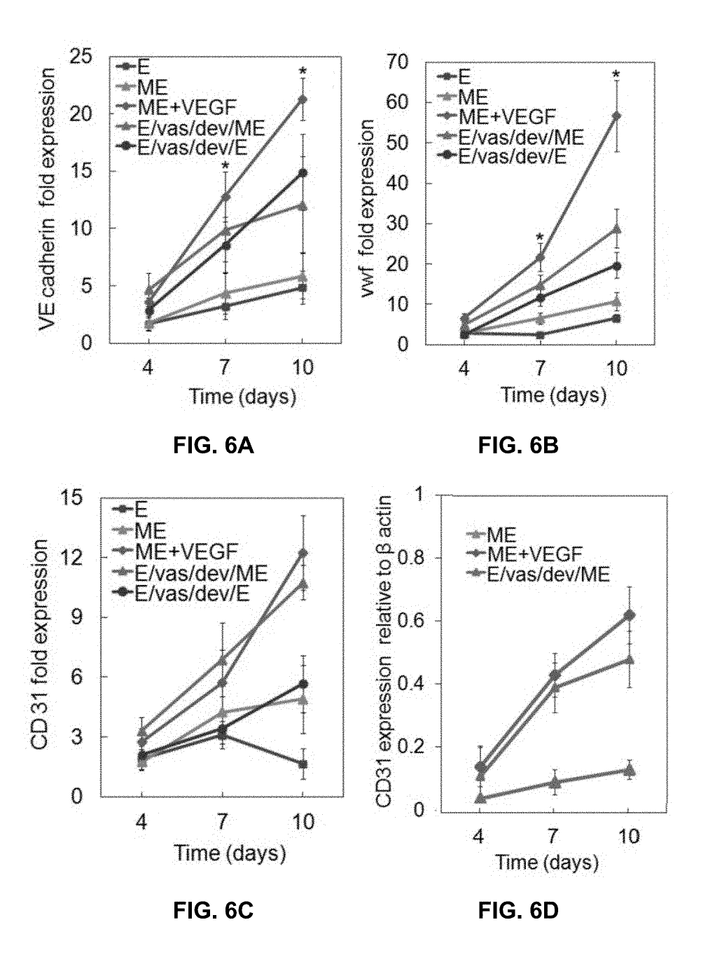

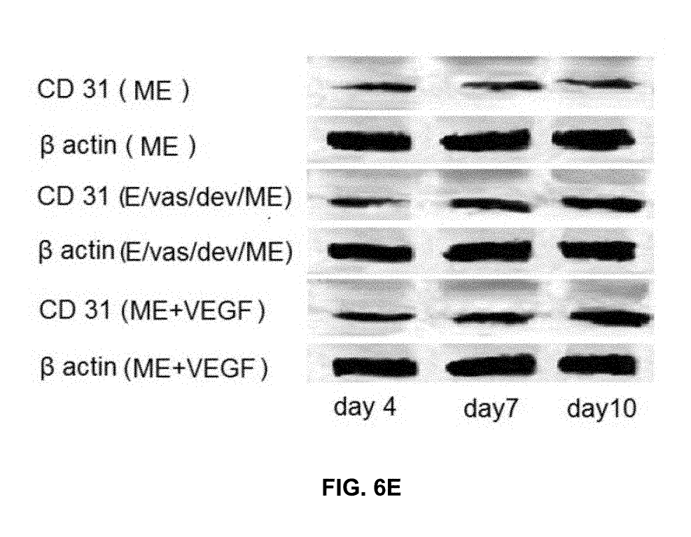

The effect of devitalized cells on vasculogenic differentiation was evaluated by reseeding ECFCs or hMSCs+ECFCs on devitalized cell sheets and culturing in basal vasculogenic medium (no VEGF) for 7 days. Results are shown in FIG. 6A-FIG. 6E. Groups included mineralized microsheets seeded with ECFCs and cultured in basal vasculogenic medium (E, control); microsheets seeded with hMSCs+ECFCs and cultured in basal vasculogenic medium without (ME, control) and with (ME+VEGF, positive control) VEGF supplementation; microsheets seeded with ECFCs, cultured in vasculogenic medium for 7 days, devitalized, and reseeded with ECFCs (E/vas/dev/E, experimental group) or hMSCs+ECFCs (E/vas/dev/ME experimental group) and cultured in basal vasculogenic medium. An asterisk in (FIG. 6A-FIG. 6D) indicates a statistically significant difference (P<0.05) between the test group and all other groups for the given time point. Error bars correspond to means±1 SD for n =3.

mRNA expression of vasculogenic markers VE cadherin, vWF, and CD31 as well as CD31 protein expression are shown in FIG. 6A-6D, respectively. Representative western blots corresponding to CD31 protein expressions are shown in FIG. 6E. As shown, mRNA expression of vasculogenic markers and CD31 protein expression for all groups increased with incubation time (except for E at day 10). For all time points, hMSC+ECFC seeded microsheets cultured in complete vasculogenic medium (ME+VEGF) had the highest expression of vasculogenic markers whereas ECFC and hMSC+ECFC seeded microsheets cultured in basal vasculogenic medium (E and ME) had the lowest expressions. Vasculogenic marker expressions of ECFCs (E/vas/dev/E) or hMSCs+ECFCs (E/vas/dev/ME) seeded on differentiated devitalized ECFC-seeded microsheets was significantly higher than E and ME groups but lower than ME+VEGF. For example, CD31 mRNA fold expressions of E, ME, E/vas/dev/E, E/vas/dev/ME, and ME+VEGF were 1.7, 4.8, 5.8, 9.6, and 12, respectively, after 10 days of incubation and CD31 protein expressions (relative to β-actin) for ME, E/vas/dev/ME, and ME+VEGF were 0.12, 0.47, and 0.62, respectively. Further, the expression of vasculogenic markers for hMSCs+ECFCs (E/vas/dev/ME) seeded on differentiated devitalized ECFC-seeded microsheets was higher than ECFCs (E/vas/dev/E).

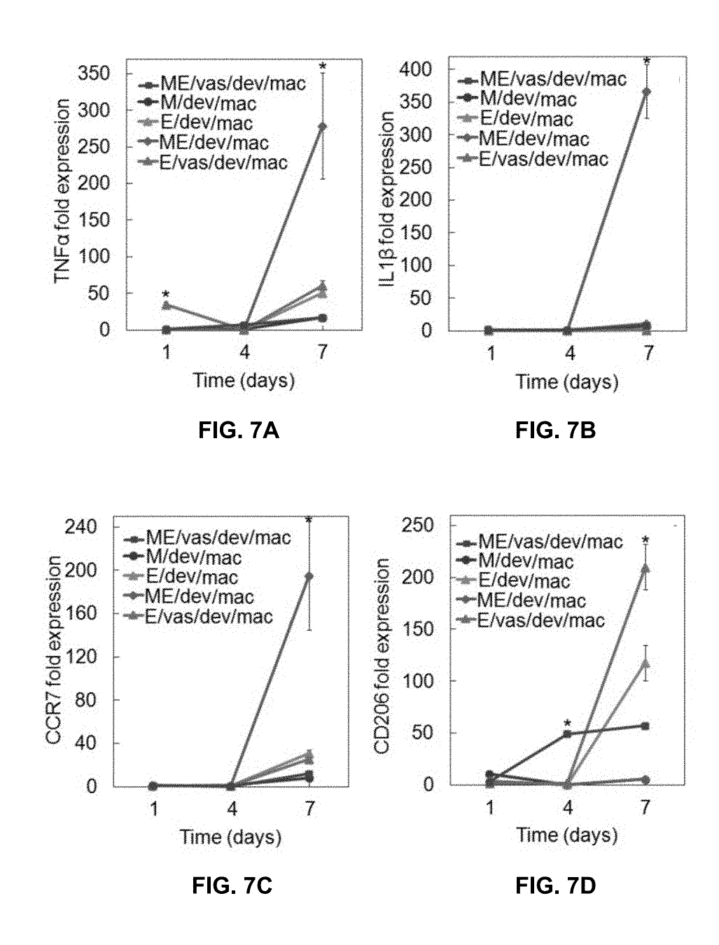

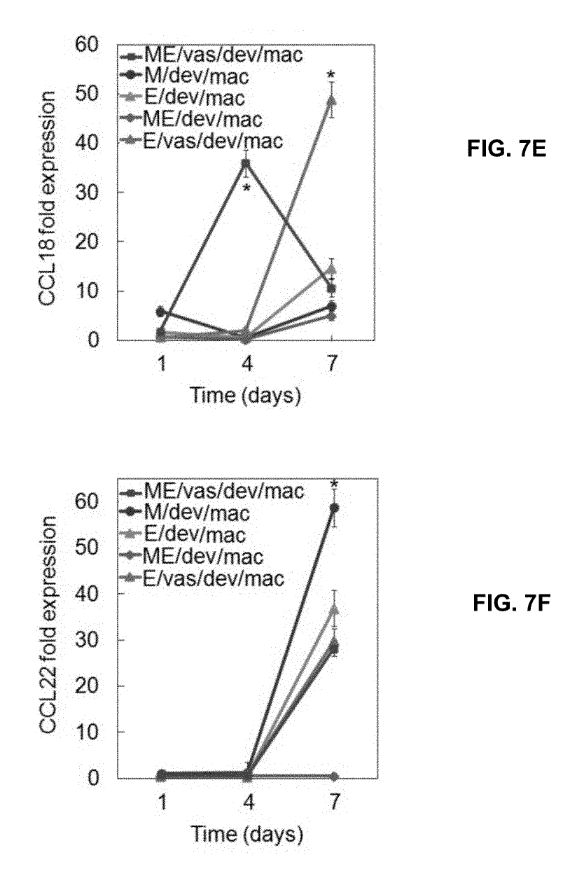

The effect of devitalized cells on macrophage polarization was evaluated by seeding macrophages on devitalized cell microsheets, culturing in basal macrophage medium, and measuring the expression of markers for M1 and M2 phenotypes with incubation time. Results are shown in FIG. 7A-FIG. 7F. Groups included mineralized microsheets seeded with hMSCs, cultured in basal DMEM medium for 1 day, devitalized, and reseeded with macrophages (M/dev/mac); microsheets seeded with ECFCs (E/dev/mac) or hMSCs+ECFCs (ME/dev/mac), cultured in basal EGM-2 medium for 1 day, devitalized, and reseeded with macrophages; microsheets seeded with ECFCs (E/vas/dev/mac) or hMSCs+ECFCs (ME/vas/dev/mac), cultured in vasculogenic medium for 7 days, devitalized, and reseeded with macrophages and cultured in basal macrophage medium for 7 days. An asterisk indicates a statistically significant difference (P<0.05) between the test group and all other groups for the given time point. Error bars correspond to means±1 SD for n=3.

For all time points, undifferentiated, devitalized hMSC microsheets and vascular-differentiated, devitalized hMSC+ECFC microsheets had the lowest gene expression of M1 markers (FIG. 7A-FIG. 7C) whereas undifferentiated, devitalized hMSC+ECFC microsheets had the lowest gene expression of M2 markers (FIG. 7D-FIG. 7F). Vascular-differentiated, devitalized ECFC microsheets showed an initial increase in the expression of TNFα M1 marker at day 1 (FIG. 7A) but undifferentiated, devitalized hMSC+ECFC microsheets showed highest final expression of all M1 markers after 7 days incubation (FIG. 7A-FIG. 7C). Conversely, vascular-differentiated, devitalized ECFC microsheets showed highest expression of M2 markers after 7 days (FIG. 7D-FIG. 7F) followed by undifferentiated, devitalized ECFC microsheets, and vascular-differentiated, devitalized hMSC+ECFC microsheets. In general, the undifferentiated, devitalized hMSC+ECFC microsheets induced polarization of macrophages to pro-inflammatory M1 phenotype whereas the vascular-differentiated, devitalized ECFC microsheets induced polarization to anti-inflammatory, pro-healing M2 phenotype.

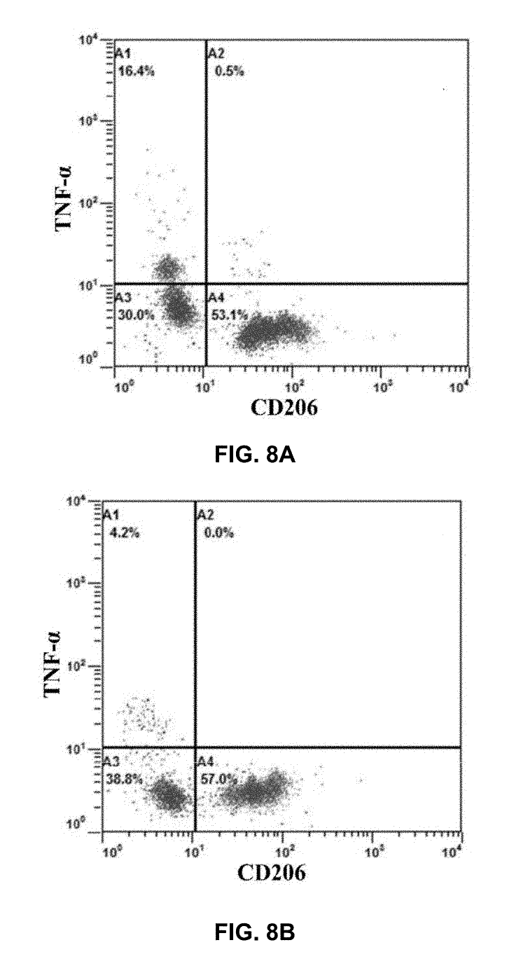

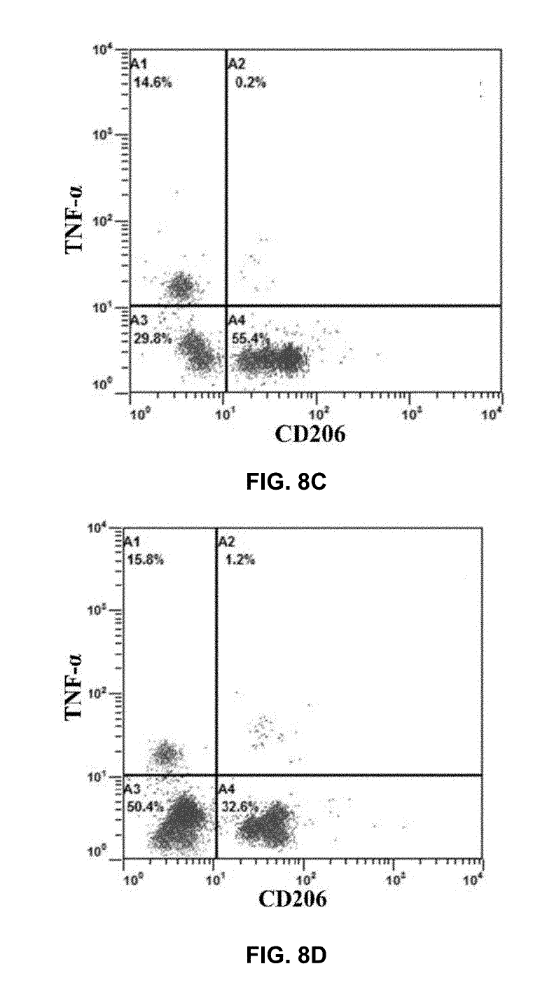

Dot blots obtained by FACS for the proportion of TNF-α (M1) to CD206 marker expressions for the macrophages seeded on devitalized cell microsheets and cultured in basal macrophage medium are shown in FIG. 8A-FIG. 8D. The devitalized cell sheets used for macrophage seeding included undifferentiated hMSCs after 1 (FIG. 8A) and 7 (FIG. 8B) days incubation in basal macrophage medium as well as vascular-differentiated ECFCs after 1 (FIG. 8C) and 7 (FIG. 8D) days.

The ratio of M2/M1 was determined by dividing the number of cells in A4 quadrant by those in A1 quadrant. M1 polarization of macrophages seeded on devitalized hMSC sheets decreased from 16% to 4% from day 1 to 7, respectively, and M2 did not changed significantly (from 53% to 57%) which led to an increase in M2/M1 ratio from 3.2 to 13.6 (FIG. 8A, FIG. 8B); M1 polarization on vascular-differentiated, devitalized ECFC sheets did not change significantly with incubation time (from 15% to 16%) whereas M2 decreased from 55% to 33% which decreased slightly M2/M1 ratio from 3.8 to 2.1 (FIG. 8C, FIG. 8D). In general, devitalized hMSC microsheets did not affect M2 macrophage polarization with incubation time while vascular-differentiated, devitalized ECFC microsheets did not affect M1 polarization. Notably, both groups had higher percentage of M2 macrophage phenotype compared to M1.

While certain embodiments of the disclosed subject matter have been described using specific terms, such description is for illustrative purposes only, and it is to be understood that changes and variations may be made without departing from the spirit or scope of the subject matter.

What is claimed is:

1. A biomimetic scaffold comprising a porous synthetic substrate and a plurality of devitalized cells incorporated on or in the porous synthetic substrate.

2. The biomimetic scaffold of claim 1, wherein the porous synthetic substrate comprises fibers.

3. The biomimetic scaffold of claim 1, wherein the porous synthetic substrate comprises an electrospun fibrous sheet.

4. The biomimetic scaffold of claim 3, wherein the porous synthetic substrate comprises a plurality of layered electrospun fibrous sheets.

5. The biomimetic scaffold of claim 1, wherein the porous synthetic substrate comprises calcium and/or phosphate minerals.

6. The biomimetic scaffold of claim 1, the devitalized cells comprising stem cells.

7. The biomimetic scaffold of claim 1, the devitalized cells comprising mesenchymal stem cells.

8. The biomimetic scaffold of claim 1, the devitalized cells comprising endothelial colony forming cells.

9. The biomimetic scaffold of claim 1, wherein the biomimetic scaffold is sized and configured for use as a bone filler or bone chips.

10. A method for forming a biomimetic scaffold comprising:
seeding living cells on a porous, synthetic substrate;
culturing the living cells for a period of time following the seeding; and
devitalizing the living cells following the period of time.

11. The method of claim 10, further comprising forming the porous synthetic substrate.

12. The method of claim 10, wherein the living cells comprise stem cells.

13. The method of claim 12, wherein the step of culturing the living cells comprises differentiating the stem cells.

14. The method of claim 10, wherein the step of devitalizing the living cells comprises lyophilization of the living cells.

15. The method of claim 10, further comprising shaping the porous, synthetic substrate.

16. The method of claim 10, wherein the biomimetic scaffold is configured for implantation in a living being.

17. The method of claim 16, wherein the living cells are autogenic cells.
